(19) United States
(12) Patent Application Publication
(10) Pub. No.: US 2004/0254590 A1
(43) Pub. Date: Dec. 16, 2004

(54) Method and instrument for the performance of stapled anastamoses

(76) Inventors: Gary H. Hoffman, Beverly Hills, CA (US); Richard B. Kaplan, Beverly Hills, CA (US)

Correspondence Address: Charles H. Schwartz, 9350 Wilshire Blvd. # 206, Beverly Hills, CA, 90212, US

(21) Appl. No.: 10/864,613

(22) Filed: Jun. 9, 2004

Related U.S. Application Data
(60) Provisional application No. 60/478,910, filed on Jun. 16, 2003.

(51) Int. Cl. A61B 17/10

(52) U.S. Cl. 606/139; 606/153

(57) ABSTRACT

An apparatus and method for performing a surgical procedure on first and second adjacent circular portions of mucosa of tubular tissue wherein a first supporting member supports the first circular portion of mucosa, a second supporting member supports the second adjacent circular portion of mucosa and with a body tubular tissue holder positioned on the first or second supporting members. The body tubular tissue holder has a plurality of holding members oriented to hold the first or second adjacent circular portions of mucosa to secure the first and second adjacent portions of mucosa to provide for attachment of the first circular portion of mucosa to the adjacent second circular portion of mucosa.

This application claims the benefit of U.S. Provisional Application No. 60/478,910, filed Jun. 16, 2003.

### DESCRIPTION

1. Field of the Invention

Hemorrhoidal surgery and anorectal prolapse operations have evolved from hand-sewn surgical hemorrhoidectomies and various hand-sewn prolapse repairs to mechanically-stapled hemorrhoidectomies and mechanically-stapled prolapse operations using a circular stapler and various pieces of supporting instrumentation. These operations have various names, but are commonly called “Procedure for Prolapse and Hemorrhoids” (PPH), Stapled Hemorrhoidopexy or Stapled Hemorrhoidectomy. The present invention and technique relates, in general, to circular staplers and circular stapling techniques and, more particularly, to the use of a mucosal fixation device to allow a more rapid and reliable surgically stapled hemorrhoidectomy and hemorrhoid removal procedure, as well as a more rapid and reliable surgically stapled rectal prolapse removal or repair. It also relates to procedures involving circular stapled anastamoses performed on other bodily tissues. This invention has applicability to other gastrointestinal procedures as well. These include various intestinal resections, repairs of rectoceles, and treatment of rectal procidentia (the prolapse of the entire rectum). The field is limited only by the skill and experience of the operator. A surgeon skilled in the art may find other uses for the device and technique.

2. Background of the Invention

Hemorrhoids are naturally occurring veins and pools of veins surrounding the anal canal. They are located just proximal to the anus. In normal existence, hemorrhoids may act as a “bumper” or cushion aiding and easing the passage of stool through the anal canal. When enlarged (as caused by chronic straining, constipation, childbirth or other unknown genetic, hereditary or environmental factors), symptoms may result. These symptoms may include bleeding, pain, hemorrhoidal prolapse (protrusion of the hemorrhoids through the anus to the exterior), itching or other symptoms. As these veins become further damaged, the flow of blood through them (returning to the heart) can slow, causing additional injury. Oftentimes, this can lead to thrombosis. Thrombosis is a condition whereby a blood clot forms in the vein and stretches the vein even further. These can be excruciatingly painful. Conservative forms of treatment have ranged from topical medication and bed rest to stool softeners and oral pain medication. As the process and the disease evolve, an operation may be required to remove and obliterate this disease. As the hemorrhoids worsen in severity, the operation to remove them may become more complex, more painful and the recovery period prolonged.

Internal hemorrhoids are classified in four degrees. Second, third and fourth degree hemorrhoids are severe and involve some measure of prolapse (extrusion from the anal canal). For these, operative intervention may be necessary.

There has been an evolution in the surgical treatment of internal hemorrhoids. Closed hemorrhoidectomy, as popularized by Ferguson, involves the use of a Ferguson-Hill retractor to obtain exposure of the hemorrhoids. The hemorrhoids are then clamped and the vessels are ligated. The hemorrhoid is dissected from the rectal wall. The surgical site is then sutured closed. This procedure is repeated until the offending hemorrhoids are removed. The post-operative course is usually extremely painful.

A similar procedure (performed most commonly in Europe) involves an open or Milligan-Morgan hemorrhoidectomy. In this procedure, using clamps, the hemorrhoids are pulled down and a second clamp is applied to the main hemorrhoidal mass to produce a “triangle of exposure”. Following this, the hemorrhoid is dissected from the underlying sphincter muscle and is dissected proximally and ligated. The wound remains open. The post-operative course is usually extremely painful

In both of these traditional hemorrhoidectomies, the pain can be intense enough such that patients prefer to coexist with their symptoms rather than undergoing an operative procedure.

Dr. Antonio Longo addressed this issue with his development of a circular hemorrhoidal stapler and accompanying anal dilator, obturator, purse string anoscope and purse-string suture puller (U.S. Pat. Nos. 6,083,241, 6,102,271 and 6,142,933), and the descriptions in his patents are incorporated by reference in this application. His method and instrumentation was based upon the previous use of circular staplers in bowel resective surgery. It was also an advance based upon a method developed by Dr. G Allegra and presented in his 1990 paper entitled “Particular Experience with Mechanical Sutures: Circular Stapler for Hemorrhoidectomy”. This was published in Giom Chir. Vol. 11-No. 3-pp 95-97, March 1990. The procedure, as described, involved gathering the rectal hemorrhoidal tissue into the center chamber of a circular stapler. The stapler was closed and fired and the hemorrhoidal tissue was removed. The remaining free ends of the rectal lining (and only the lining; not the deeper layers of tissue) were then automatically reattached to each other during the process, using preloaded staples. A key step in the initial part of the procedure was the placement of a circumferential purse string suture in the rectum. This purse string suture was designed to gather only superficial hemorrhoidal tissue (arteries, veins, and rectal mucosal and submucosal lining) into the center of the stapler chamber, thus allowing the hemorrhoidal tissue to be cut out by the stapler. The deeper layers of the rectal wall remained intact and unaffected by the action of the stapler.

These mechanical staplers previously had been used in colon resective to perform extremely low (deep in the pelvis) anastomoses, which heretofore had been difficult or impossible to perform using a hand-sewn technique. An example of one such stapling device is the ECS 25 Endopath, TM, ILS Endoscopic Curved Intraluminal Stapler from Ethicon Endo-Surgery, Inc., Cincinnati, Ohio. Many circular stapler patents and patents that illustrate procedures that use circular staplers now exist; for example, U.S. Pat. No. 4,207,898 by Becht, U.S. Pat. No. 4,592,354 by Rothfuss, U.S. Pat. No. 5,122,156 by Granger et al, U.S. Pat. No. 4,351,466 to Noiles, U.S. Pat. No. 5,292,053 to Bilotti et al, U.S. Pat. No. 5,344,059 to Green et al, and U.S. Pat. No. 6,117,148 by Ravo et al.

There are also devices designed to remove transmural sections of tissue. Examples of such a resectioning device are shown in U.S. Pat. No. 5,868,760 by McGuckin Jr. and U.S. Pat. No. 6,264,086 by McGuckin Jr. This type of resection device is different than the present invention since it is limited to the transmural holding of tissue (the entire, full thickness of intestine; i.e. all tissue layers).

Intestinal tissue is made up of several distinct tissue layers. The innermost layer is the mucosa followed by (from inside to outside) the submucosa and blood supply, the muscular layer and the outer lining or serosa (this serosal layer is absent in the rectal wall however, and is replaced by a layer of fibrofatty connective tissue).

Traditionally, surgical staplers have been used for operations upon the entire thickness of a given tissue. This has been true for linear staplers that both cut (transect) and then staple together the full thicknesses of two separate tissues (usually intestinal). It has also been the case with circular staplers which join (anastamose) two full thickness circular structures (usually intestinal) to each other.

Simply stated, the diseased piece of intestine is removed and the resulting free ends are anastomosed to each other. Prior to the invention of surgical staplers, this anastamosis was performed using hand-sewn techniques. This sewing was particularly difficult when performing anastamoses deep in the pelvis. Safe suturing was hampered by poor lighting and visualization, inadequate instrumentation, and difficult operative field exposure. The introduction of surgical staplers allowed for safer, more rapid and even deeper resections and anastamoses. Again, however, the lack of an easily and reliably reproducible step, the placement of a purse string suture to secure the full thickness of the intestinal tissue around the center rod of the stapler, greatly hindered the process. Less reliable and more time-consuming hand-sewn techniques were used. The hand-sewn technique was particularly difficult to use in deep pelvic operations where lighting, visualization and small spaces were involved.

During the performance of the anastamotic phase of the operation, whereby the full thicknesses of each of the two severed ends of the tubular, hollow intestine are joined permanently to each other, the head of the stapler (the anvil) must be placed into one end of the intestine, and the working end of the instrument must be placed into the other end of the intestine. (This requires the surgeon to be able to actually hold each end of the opened intestine or other tubular structure). Both ends of the intestine must be secured around the anvil and also around the chamber end of the stapler while these ends are brought together and the machine is securely closed and fired. It should be noted that this procedure is performed with the patient's body cavity opened either surgically or laparoscopically. Nevertheless, all steps of the operation are performed whilst the surgeon has complete access to the external portion of the intestine (the serosal layer).

It is at this step (the placement of the anvil and it's matching opposite end) where the purse string suture placement is critical. The purse string suture must secure the entire thickness (all of the previously described layers) of the intestine. If any layer of intestine is excluded from the purse string suture, an inadequate anastamosis will be performed, leading to leakage of intestinal contents and to potential surgical disaster and loss of life. This placement of this suture is a critical step in the operation. It ensures that the entire thickness of the intestine is secured both around the anvil and center rod, and separately around the chamber end of the instrument and center rod of the stapler. The focus of the surgical effort is on the entire, full thickness of the intestinal tissue.

The purse string can be placed using a hand-sewn technique. However, this is difficult and time consuming. As an alternative to hand sewing, automated purse string applicators have been developed as shown by U.S. Pat. Nos. 4,749,114, 4,821,939 and 5,158,567 (United States Surgical Corporation, Norwalk, Conn.).

Within the last several years, a field of surgery has emerged which has concerned itself with the joining together of only the inner most lining (i.e. mucosa and submucosa) of two structures, using surgical staplers. The focus of the surgical effort here, in contradistinction to the above referenced traditional intestinal stapled or hand sewn operations, is on only the most superficial, mucosal, submucosal and vascular layers of the rectum and hemorrhoidal tissue.

The emergence of stapled hemorrhoidectomy (or stapled hemorrhoidopexy), and stapled rectal prolapse repair are but two examples of a field of operations centered around, and concerned with this innermost layer of tissue. A significant difficulty in these operations in general, and stapled hemorrhoidectomy and prolapse repair in specific, has been the placement of a circumferential inner layer stitch (purse string suture), to adequately hold, or fix only the inner layer in place whilst the surgical stapler performs a cutting and joining function on this layer, and only on this layer. (The other layers of tissue are unaffected and are left undisturbed).

Importantly, Dr. Longo recognized “the need for a simple and fast method of performing a hemorrhoidectomy” Dr. Longo's work in modifying the circular stapler for hemorrhoidal removal was based upon the observation that hemorrhoidal surgery performed above (proximal to) the dentate line (that anal anatomic landmark differentiating internal from external hemorrhoids) was significantly less painful than the traditional operation. It was felt that this relative lack of postoperative pain was related to the scarcity of pain fibers (nerves) above the level of the dentate line. To that end, Dr. Longo advanced a technique whereby the hemorrhoidal tissue was removed in a circumferential fashion from above (proximal to) the dentate line, and the remaining rectal lining was re-anastomosed. This was performed using a circular stapler. Furthermore, Dr. Longo advanced the use of a circular anoscope (dilator) with a central obturator through which this hemorrhoidal stapler could be placed. Prior to placing the instrument, however, Dr. Longo believed that a method whereby the rectal mucosa could be drawn into the stapler had to be perfected. To do this, Dr. Longo advanced the use of a purse-string anoscope which, when placed into the anal canal through the circular anal dilator, allowed the operating surgeon to place a purse-string stitch. This purse-string stitch could then be used to bring the mass of hemorrhoidal arteries and veins and tissue into the center chamber of the hemorrhoidal stapler, thus allowing it to be transected during the operation of the instrument. The placement of this purse string stitch was the key step and advance in allowing the stapled hemorrhoidectomy to be performed.

The limitation of this procedure, as conceived by Dr. Longo, is that the placement of the purse-string suture is an awkward, cumbersome and time-consuming technique. It can oftentimes yield erratic results and lead to well known surgical complications. Its placement is difficult to safely reproduce from patient to patient. Due to the proximal location of the purse-string, (high in the rectum), the area can sometimes be inadequately lit, precluding adequate visualization of the placement of the purse-string. Additionally, placing the purse-string at an appropriate depth is also inconsistent. In cases of severe hemorrhoids, visualization of the appropriate location for each purse-string bite is very difficult.

Often, the purse-string is placed elliptically rather than in a circular orientation. This yields an inconsistent size and shape of excised anorectal tissue.

Whereas Dr. Longo did indeed refine and popularize the use of the circular surgical stapler and anoscope for the performance of stapled hemorrhoidectomy, the procedure had a major limitation; namely the difficulty of use and correct placement of the purse string suture This has understandably slowed its adoption into widespread use. The actual placement of the suture was time consuming, difficult, ergonomically awkward and potentially dangerous. Often, the purse string suture was, and still is inadvertently and unavoidably placed elliptically rather than in a circular orientation. This yields an inconsistent size and shape of excised anorectal tissue. Frequently, the purse string suture is placed too deep into the rectum, and includes too many layers of tissue, resulting in a full thickness resection and potential surgical complications.

Additionally, the technique of placing the purse-string suture is time-consuming, requiring considerable skill and experience, and adding up to 15 to 25 minutes to the procedure.

Finally, it has been found that the placement of the purse-string in the performance of a circular stapled hemorrhoidectomy is the single factor slowing the adoption of this technique. Surgeons rightly express the concern that the purse-string cannot be placed correctly, safely, adequately or in the proper anatomical location. Fear of incorrect placement of the purse-string suture has caused many surgeons to continue to perform traditional, painful open hemorrhoidectomies, or, to abandon the use of the stapled procedure after a difficult learning period.

Much attention has been given to inventing a mechanical technique for the more rapid and safe placement of the purse string. In traditional intestinal surgery, there are many examples of purse string devices that allow a purse string suture to be mechanically placed around the most outer layer (the serosal layer) of the intestine. Automated purse string applicators have been developed as shown by U.S. Pat. Nos. 4,749,114, 4,821,939 and 5,158,567 (United States Surgical Corporation, Norwalk, Conn.).

When secured, the externally placed purse string secures all tissue layers (“full thickness”) around the stapler center rod and into the chamber of the stapler. However, this technique of securing the entire thickness of the intestinal tissue has no applicability when performing a stapled hemorrhoidectomy. This traditional, full thickness purse string technique is performed with the abdomen fully opened and the intestines fully exposed whilst the surgeon holds the intestines (or other tubular structure) in his hands. This is a full thickness gathering of the intestinal tissue. This has no applicability in operations upon only the inner layers of tissue (as in rectal or hemorrhoidal operations) and cannot be used in these types of operations.

There are also documented methods for attempting to hold the full thickness of the intestine in place using spikes or impalement techniques, as shown by U.S. Pat. No. 5,122,156 issued to Granger et al. While these spikes are placed from within the intestine, they still require complete access (for manipulation) to the external, serosal aspect of the intestines. They too are designed to be placed using an opened abdomen (surgically or laparoscopically). Importantly, these are still full thickness techniques, designed for a full thickness operation. Additionally, in one embodiment of the procedure, additional sutures are still shown as being required to hold the intestinal ends in place over the impalement spikes.

With the development of the stapled hemorrhoidectomy, attention focused on the resection (removal) of only a superficial inner layer of rectal hemorrhoidal tissue. Indeed, the development of the operation hinged upon this step of the procedure. A full thickness tissue operation was both not required and was undesirable. Additionally, the procedure had to be performed in a closed space, whereby the surgeon had access to only the inner layers of the intestine (in this case the rectum). The surgeon no longer was able to hold the entire tubular structure or control the placement of the purse string suture.

Traditional hemorrhoidectomy was an exquisitely painful operation. The great advance in the stapled technique was that it was performed in an area of the rectum that has very few pain fibers, thus allowing for an almost painless hemorrhoidectomy. It was recognized early on that the potential for severe and dangerous complications existed if the full thickness of the rectum was removed or if the purse string suture was placed in the wrong anatomical location. Hence, close attention was given to the correct and safe placement of the superficial purse string suture. However, this was, and remains a technically difficult and time-consuming step in the procedure.

In our early work with the procedure, we began to address this vexing problem. Initially we (and others) searched for better ways to place the purse string suture. Through a series of discoveries, we concluded that were able to remove the need for the placement of a purse string suture and secure the inner rectal lining around the stapler center rod with a more reliable, safer, and reproducible method and device. Therefore, we devised an apparatus and method to completely and safely remove this step of the operation. We discovered a method and apparatus to hold only the mucosa, submucosa and hemorrhoidal vessels securely in place during the mechanical performance of the cutting and stapling portion of a stapled hemorrhoidectomy. The remainder of the rectal layers (the muscle and fibrofatty tissue) are left undisturbed. Additionally, the technique is done with direct visualization of the entire operative field (the inside of the rectum). Finally, it is performed from inside the rectum, with the surgeon having no need for access to the outer rectal tissues (as in traditional intestinal resective operations). The placement and use of the purse string stitch is completely eliminated.

In essence utilizing our apparatus and method, we have removed the need to place a purse string suture, thus removing a difficult, potentially dangerous and time-consuming portion of the operation.

What is required, and what we have invented is a method of rapidly, safely, reliably and reproducibly securing the hemorrhoidal tissue and rectal mucosa in place so that transection and anastomosis can be performed using the stapler. This technique should be safe, straightforward and easy to learn. This technique should reduce the amount of time necessary to complete the stapled hemorrhoidectomy. What is needed is a fixation device that removes the variability in this procedure and allows a more reliable and consistent fixation.

The foregoing is also applicable to operative procedures for removal or repair of anorectal prolapse, as well as other gastrointestinal procedures, gynecological, vascular and other procedures.

At present, there are no known surgical tools or techniques that can meet these needs. These and other advantages will become more apparent from the following detailed description and drawings.

### SUMMARY OF THE INVENTION

A method and instrument to allow Stapled Hemorrhoidectomies (or any other mechanically stapled procedure for prolapse or hemorrhoids as well as other gastrointestinal, gynecological, vascular and other procedures) to be performed in a more rapid and expeditious fashion, allowing the surgeon to more uniformly perform the procedures is disclosed. This method involves the use of a new form of mechanical stapler that includes a device used to hold the anorectal mucosa in place during the stapling procedure. Said device contains a multiplicity of spines or gripping members that impale or grip the mucosa and hemorrhoidal vessels and hold them in place around the stapler center rod so that the mechanical stapler can then remove the necessary tissue and perform an anastamosis on the remaining lining (mucosa).

What is described herein is a device with a multiplicity of spikes, spines, or surfaces capable of holding or gripping the anorectal or gastrointestinal mucosa and submucosa and vessels (or other types of mucosa) in a fixed position and location while performing a stapled hemorrhoidectomy, prolapse repair or other procedure.

### The Longo Procedure

Longo U.S. Pat. Nos. 6,102,271 and 6,142,933 and 6,083,241 show the traditional or Longo stapled hemorrhoidectomy that proceeds as follows:

A circular anal dilator and obturator are inserted into the anal canal. Once in position in the anal canal, the obturator is removed and the anal dilator is held in place with several sutures placed through the openings in an attached flange.

With the surgeon standing on the left side of the patient, a purse string anoscope is then inserted into the anal canal. The surgeon begins the purse string suture placement in the left anterior quadrant of the patient. A circumferential mucosal/submucosal purse string suture is placed approximately 4 cm-6 cm proximal (above) to the dentate line (that area of the anal canal where the pain fibers terminate).

At this point, a hemorrhoidal stapler is opened to its maximal extent. Its anvil is placed into the rectum. It is carefully, and under direct vision, advanced proximal to the purse string. When it has been determined that the anvil is indeed located proximal to the purse string suture, the purse string suture is cinched closed around a center rod of the stapler. Visual and tactile inspection will ensure that the entire circumference of the anorectal mucosa is snug around the center rod. The purse string suture is then tied. The ends of the suture are now brought out through the side of the instrument using a purse string puller,

The first assistant applies gentle outward traction on the purse string suture, while the surgeon gently pushes the instrument into the anal canal and closes the stapler jaws. Gentle traction is maintained on the suture tails as the instrument is closed.

The surgeon then removes the circular anal dilator and everts the perianal buttock skin. This important step is done to ensure that the dentate line is actually seen to be free from the jaws of the stapling device.

The stapler is fired and held closed for two or three minutes to aid in hemostasis. Finally, the stapler is opened, and removed and the staple line is visualized. For additional information on the Longo procedure, the following article appearing on pages 489-499 in the June issue of “Surgical Rounds” entitled “Hemorrhoids—To staple or not to staple: that is the question” is incorporated by reference. The authors of this article are Gary H. Hoffman MD and Eiman Firoozmand MD.

As can be seen from the prior art, the key step is the safe and careful placement of the purse string suture. Placed too deep, and too much tissue will be pulled into the stapler possibly leading to a surgical misadventure.

### THE PRESENT INVENTION

In the present invention, all steps relating to the placement and activation of the purse string suture are eliminated from the procedure thus decreasing operative time by approximately 15 to 25 minutes, and allowing for a safer, more reliable, reproducible and rapid procedure.

The initial performance of a stapled hemorrhoidectomy or prolapse repair as provided by the present invention is the same as provided for in the Longo procedure. Initially, the buttocks are taped in the spread-apart position. With the patient suitably anesthetized, the anal obturator is placed into the clear, circular anal dilator. The combined apparatus is then placed into the anal canal. Once in position, the obturator is removed and sutures are then placed into the slots provided on the anal dilator flange to hold the anal dilator in position.

At this point, the procedure of the present invention departs from the traditional procedure as described by Longo.

The stapler is equipped with a fixation device. Or, the device can be manufactured separately from the stapler, and placed onto the center rod of the stapler during the conduct of the operation.

The device consists of a multitude of spike like protuberances which are affixed to a washer which is placed onto and around the center rod of the stapler (and facing either the anvil or the chamber end of the stapler), such that the fixation device will impale and retain the mucosa in a suitable position while the jaws of the cylindrical stapler are closed. Alternatively, the spikes may be affixed circumferentially to the anvil of the instrument and point toward the larger, chamber end of the stapler. Alternately, the spikes are affixed circumferentially to the larger, chamber end of the instrument, pointing toward the anvil. Other fixation, gripping or holding devices may be used in substitution for the spikes.

To begin the procedure the stapler is opened to its maximum extent. The anvil is carefully inserted through the cylindrical anoscope into the anorectum. The anvil is advanced to a point approximately 4 to 6 cm proximal to the dentate line. At this stage, the anorectal mucosa billows inward and actually covers the retention device. Additionally, if this inward billowing of the mucosa is not complete or is inconsistent, the mucosa can be placed onto the spikes and manipulated using tissue forceps and direct visualization. If the anorectal mucosa appropriately surrounds the center shaft and fits over the spines of the retention device, the entire apparatus is gently pulled out of the anorectum for approximately 1 cm. This in turn secures the anorectal mucosa onto the spines of the device. Thus, the anorectal mucosa is secured to the fixation device. Alternately, tissue forceps can be used to gently elevate and move the mucosa further centrally (toward the center shaft) or radially (away from the center shaft) to adjust the amount of tissue trapped and fixed by the device. At this point, while exerting gentle, outward traction on the entire instrument, the instrument is closed.

The procedure now returns to the standard Longo procedure. The stapler is held closed for 30 seconds and then fired and held closed for two to three minutes to aid hemostasis.

The stapler is opened and removed from the anorectum. The area is inspected for hemostasis. A light dressing is applied and the patient is taken to the recovery room.

The doughnut of resected tissue is removed from the instrument and inspected. It is then sent to the pathologist for a complete pathological description.

### BRIEF DESCRIPTIONS OF THE DRAWINGS

FIG. 1 illustrates a first embodiment of the invention.

FIG. 2. illustrates a second embodiment of the invention.

FIG. 3 is a detailed drawing of the washer and spikes and also shows the attached retainer.

FIG. 4 is a detailed drawing of two of the many possible spikes.

FIG. 5 shows a detailed drawing of an alternate washer, spikes and retainer.

FIG. 6 is the third embodiment of the invention.

### DESCRIPTION OF THE PREFERRED EMBODIMENT

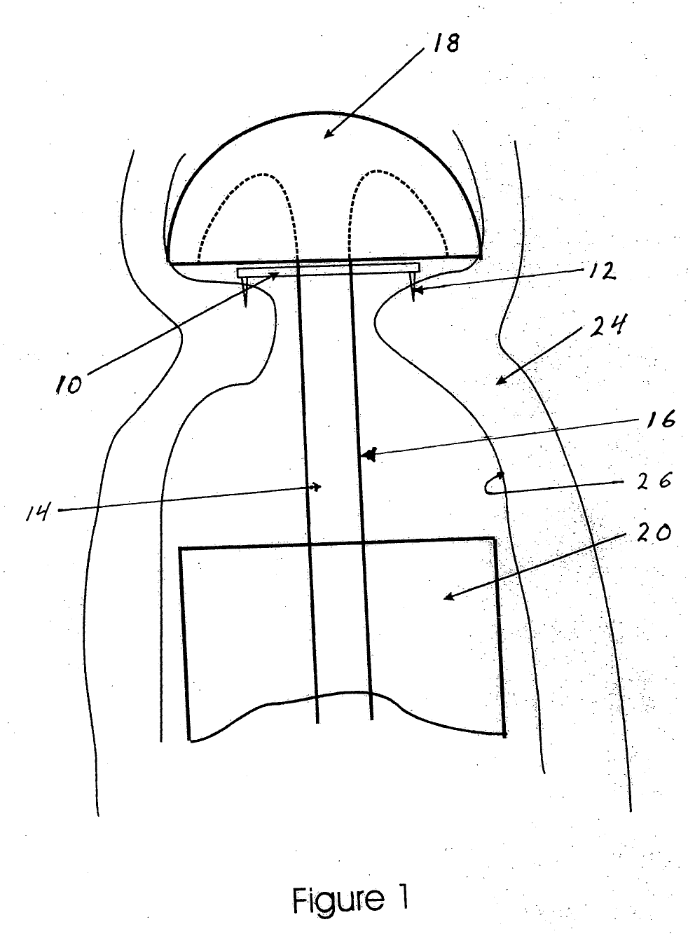

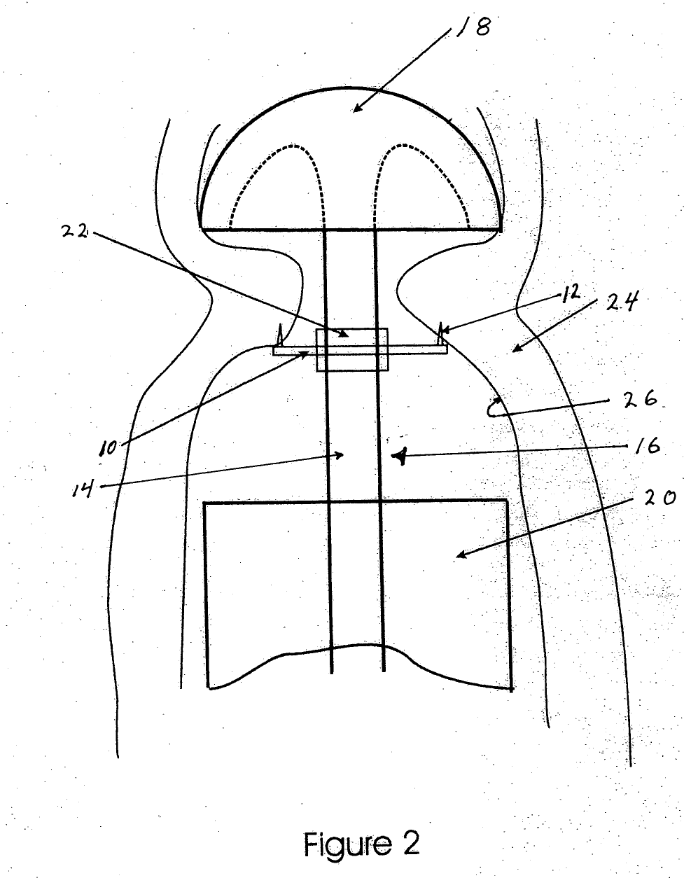

As shown in FIG. 1, a washer like device 10 having spikes 12 is affixed to the central shaft 14 of the stapler 16. The spiked washer 10 is against the stapler head (anvil) 18 and the spikes 12 face away from the stapler head 18, toward the body 20 of the stapler 16. It may or may not have an attached or separate retainer. The washer like device 10 has an outside diameter of approximately one half the inside diameter of the instrument cutter (not shown) which is contained in the stapler body 20. The washer like device 10 has the proliferation of spikes 12 protruding from its outer edge, while nominally pointing in an axial direction away from the upper anvil 18 of the stapler 16. As shown in a second embodiment of the invention in FIG. 2, the washer like device 10 is mounted on a retainer 22 and the washer spikes 12 face toward the stapler head 18. It should be noted that in the embodiment of FIG. 1, the washer 10 may have this retainer 22 as well. As shown in FIG. 1 and FIG. 2, all of the above structure is located within the tubular tissue 24 and adjacent the mucosal wall 26.

FIGS. 1 and 2 show the stapler 16 in the open position, with the spikes 12 of the washer 10 impaling and holding the superficial mucosa, submucosa and vasculature of the tubular hemorrhoidal tissue 24 in the rectum. Note that the deeper, outer layers of the tubular tissue 24 are intact, unattached to the fixation washer, and in their normal anatomic position.

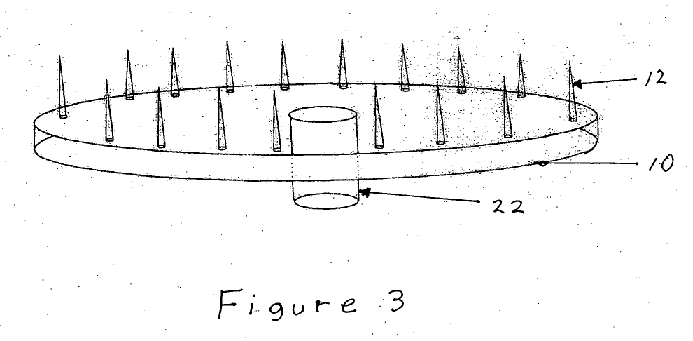

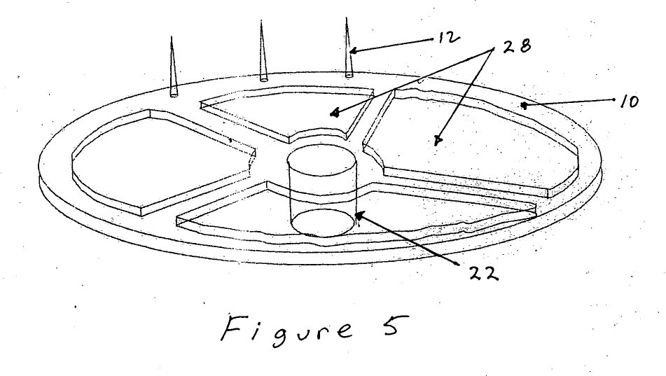

In the embodiments of FIG. 1 and FIG. 2, the individual spikes 12 are approximately 1 mm in length (as shown in detail in FIG. 3), and are sufficiently sharp enough to puncture the mucosal wall 26. The entire washer apparatus 10 can be made of suitable plastic, stainless steel or any other suitable metal or alloy. Two of the many possible washer configurations are shown in FIGS. 3 and 5. In FIG. 5 the center retainer and outside washer and spikes have intervening open areas 28 to allow for tissue manipulation during the operation.

As the inner portion of the anvil 18 is recessed, and as the center of the chamber of the stapler is hollow, the washer like device 10 could move into the anvil 18 recess or fall into the center chamber of the body 20 of the stapler 16. Therefore, the washer like device 10 may have the inner clamping ring or “bumper” retainer 22 (either attached to or separate from the washer) so that the washer 10 can be held securely in place and positioned correctly on the center rod 14 of the stapler. This ability to position the washer like device 10 can be used to control the amount of tissue that will be removed when the stapler is closed and fired. This will also fix the washer like device 10 in place and prevent movement during the conduct of the operation. Additionally, depending upon manufacturing and other considerations, the washer like device 10 can face either toward or away from the anvil 18. It should be noted that the axial placement of the washer 10 determines the length of mucosa that is removed during the cutting phase of the operation. This ability to remove various sized “doughnuts” of tissue make the present invention adaptable to removing sections of diseased mucosa and small mucosal growths

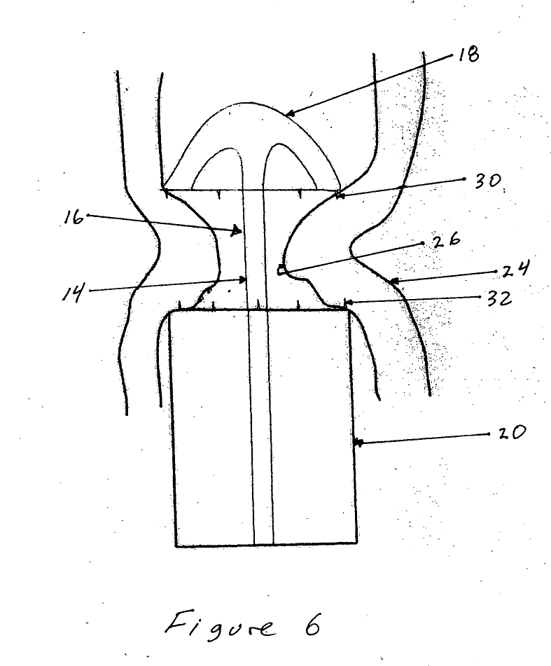

A third embodiment of the invention, shown in FIG. 6, has spikes 30 affixed permanently to the inner periphery of the staple head (anvil) 18. This embodiment additionally shows spikes 32 arrayed circumferentially around the body 20 of the stapler, and in both cases has no washer or retainer. Additionally, it is possible that the spikes 30 or 32 may be placed only on the anvil 18, or only on the body 20 of the stapler, or on both the body and anvil (as shown). This embodiment allows the mucosa to be held in place while the anvil 18 and body 20 are drawn together and closed for firing. However, it does not allow for adjustment of the amount of tissue ring that will be excised.

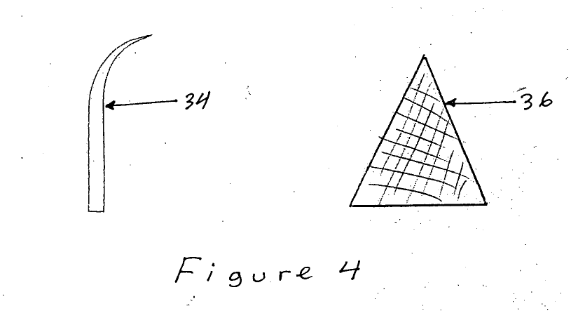

FIG. 4 illustrates two additional shapes that the spikes may take in addition to the straight spikes shown in FIG. 3. Specifically, as shown in Figure the spikes may be curved as shown by spikes 34 or triangular as shown by spikes 36.

Other variations on the invention may include fabricating the device from a series of steel spring wires or other barbed wires that are wrapped individually around the central shaft. The tips of said wires are then pointed axially and/or slightly inwardly so as to trap the anal mucosa and hold it in place while the stapler head and body are drawn together for firing. Other holding or gripping structures could include slotted discs, Velcro like materials, fishhooks and any other structures that hold or grip the inner wall of the tubular tissue.

While preferred embodiments of the present invention have been shown and described herein, it will be obvious to those skilled in the art that such embodiments are provided by way of example only. The invention discloses the spike (or spine) fixation technique and instruments, and any mechanism that accomplishes this fixation in a safe and secure fashion will be suitable.

Additionally, it has been demonstrated that this same instrument and procedure could also be used for the removal of or repair of rectal prolapse or rectoceles. It should also be noted that by adjusting the placement of the spiked retention device, the amount of mucosa may be varied. This can allow for the removal of small mucosal growths as well as healthy tissue as in the case of hemorrhoidectomies or mucosal prolapse. It should be noted that the device herein described can be used in other procedures involving the anastamosis of tubular anatomic structures. The limitations are only the ability to insert the device into the proper location and attach the tubal wall to the spiked retention spines.

What is claimed is:

1. In combination with an apparatus for performing a surgical procedure on first and second adjacent circular portions of mucosa of tubular tissue wherein a first supporting member is adapted to support the first circular portion of mucosa and a second supporting member is adapted to support the second adjacent circular portion of mucosa and to provide for attachment of the first circular portion of mucosa to the adjacent second circular portion of mucosa which comprises, a body tubular tissue holder positioned on the first or second supporting members and having a plurality of holding members and oriented to hold the first or second adjacent circular portions of mucosa to secure the first and second adjacent portions of mucosa for attachment to each other.

2. The apparatus of claim 1 wherein the first and second adjacent circular portions of tubular tissue include mucosa and submucosa and wherein the first and second supporting members and the holding members provide for attachment of the adjacent circular portions of mucosa and submucosa to each other.

3. The apparatus of claim 1 wherein the body tubular tissue holder is positioned on the first supporting member.

4. The apparatus of claim 1 wherein the body tubular tissue holder is positioned on the second supporting member.

5. The apparatus of claim 1 wherein the holding members are formed by spikes.

6. The apparatus of claim 1 wherein the body tubular tissue holder is formed as part of the first supporting member.

7. The apparatus of claim 1 wherein the body tubular tissue holder is formed as part of the second supporting member.

8. The apparatus of claim 1 wherein the body tubular tissue holder is formed separately from the first or second supporting member and includes a washer that supports the holding members.

9. The apparatus of claim 8 additionally including a retaining member connected to the body tubular tissue holder to retain the body tubular tissue holder in position relative to the first or second supporting member.

10. The apparatus of claim 1 wherein the first and second supporting members form a circular stapler to provide the attachment of the first and second adjacent portions of mucosa.

11. The apparatus of claim 10 additionally including a central shaft interconnecting the first and second supporting members and wherein the body tubular tissue holder is positioned around the central shaft.

12. In an apparatus for performing a medical procedure on first and second adjacent circular portions of mucosa of tubular tissue which comprises,
a first supporting member adapted to support the first circular portion of mucosa,
a second supporting member adapted to support the second adjacent circular portion of mucosa, means coupled to the first and second supporting members for providing for attachment of the first circular portion of mucosa to the adjacent second circular portion of mucosa, and
a body tubular tissue holder positioned on the first or second supporting members and having a plurality of holding members and oriented to hold the first or second adjacent circular portions of mucosa to secure the first and second adjacent portions of mucosa for attachment to each other.

13. The apparatus of claim 12 wherein the first and second adjacent circular portions of tubular tissue include mucosa and submucosa and wherein the first and second supporting members and the holding members provide for attachment of the adjacent circular portions of mucosa and submucosa to each other.

14. The apparatus of claim 12 wherein the body tubular tissue holder is positioned on the first supporting member.

15. The apparatus of claim 12 wherein the body tubular tissue holder is positioned on the second supporting member.

16. The apparatus of claim 12 wherein the holding members are formed by spikes.

17. The apparatus of claim 12 wherein the body tubular tissue holder is formed as part of the first supporting member.

18. The apparatus of claim 12 wherein the body tubular tissue holder is formed as part of the second supporting member.

19. The apparatus of claim 12 wherein the body tubular tissue holder is formed separately from the first or second supporting member and includes a washer that supports the holding members.

20. The apparatus of claim 19 additionally including a retaining member connected to the body tubular tissue holder to retain the body tubular tissue holder in position relative to the first or second supporting member.

21. The apparatus of claim 12 wherein the first and second supporting members form a circular stapler to provide the attachment of the first and second adjacent portions of mucosa.

22. The apparatus of claim 21 additionally including a central shaft interconnecting the first and second supporting members and wherein the body tubular tissue holder is positioned around the central shaft.

23. A method for performing a medical procedure on first and second adjacent circular portions of mucosa of tubular tissue wherein a first supporting member is adapted to support the first circular portion of mucosa and a second supporting member is adapted to support the second adjacent circular portion of mucosa and to provide for attachment of the first circular portion of mucosa to the adjacent second circular portion of mucosa which comprises the following steps,
providing a body tubular tissue holder having a plurality of holding members, positioning the body tubular tissue holder on the first or second supporting member, and
providing the plurality of holding members to be oriented to hold the first or second adjacent circular portions of mucosa to secure the first and second adjacent portions of mucosa for attachment to each other,

24. The method of claim 23 wherein the first and second adjacent circular portions of tubular tissue include mucosa and submucosa and wherein the first and second supporting members and the holding members provide for attachment of the adjacent circular portions of mucosa and submucosa to each other.

25. The method of claim 23 wherein the body tubular tissue holder is positioned on the first supporting member.

26. The method of claim 23 wherein the body tubular tissue holder is positioned on the second supporting member.

27. The method of claim 23 wherein the holding members are formed by spikes.

28. The method of claim 23 wherein the body tubular tissue holder is formed as part of the first supporting member.

29. The method of claim 23 wherein the body tubular tissue holder is formed as part of the second supporting member.

30. The method of claim 23 wherein the body tubular tissue holder is formed separately from the first or second supporting member and includes a washer that supports the holding members.

31. The method of claim 30 additionally including a retaining member connected to the body tubular tissue holder to retain the body tubular tissue holder in position relative to the first or second supporting member.

32. The method of claim 23 wherein the first and second supporting members form a circular stapler to provide the attachment of the first and second adjacent portions of mucosa.

33. The method of claim 32 additionally including a central shaft interconnecting the first and second supporting members and wherein the body tubular tissue holder is positioned around the central shaft.

34. A method for performing a medical procedure on first and second adjacent circular portions of mucosa of tubular tissue which comprises the following steps,
providing a first supporting member adapted to support the first circular portion of mucosa,
providing a second supporting member adapted to support the second adjacent circular portion of mucosa,
coupling the first and second supporting members for providing for attachment of the first circular portion of mucosa to the adjacent second circular portion of mucosa, and
providing a body tubular tissue holder positioned on the first or second supporting members and with the body tubular tissue holder having a plurality of holding members oriented to hold the first or second adjacent circular portions of mucosa to secure the first and second adjacent portions of mucosa for attachment to each other.

35. The method of claim 34 wherein the first and second adjacent circular portions of tubular tissue include mucosa and submucosa and wherein the first and second supporting members and the holding members provide for attachment of the adjacent circular portions of mucosa and submucosa to each other.

36. The method of claim 34 wherein the body tubular tissue holder is positioned on the first supporting member.

37. The method of claim 34 wherein the body tubular tissue holder is positioned on the second supporting member.

38. The method of claim 34 wherein the holding members are formed by spikes.

39. The method of claim 34 wherein the body tubular tissue holder is formed as part of the first supporting member.

40. The method of claim 34 wherein the body tubular tissue holder is formed as part of the second supporting member.

41. The method of claim 34 wherein the body tubular tissue holder is formed separately from the first or second supporting member and includes a washer that supports the holding members.

42. The method of claim 41 additionally including a retaining member connected to the body tubular tissue holder to retain the body tubular tissue holder in position relative to the first or second supporting member.

43. The method of claim 34 wherein the first and second supporting members form a circular stapler to provide the attachment of the first and second adjacent portions of mucosa.

44. The method of claim 43 additionally including a central shaft interconnecting the first and second supporting members and wherein the body tubular tissue holder is positioned around the central shaft.
